(19) United States
(12) Patent Application Publication
(10) Pub. No.: US 2026/0182902 A1
(43) Pub. Date: Jul. 2, 2026

(54) METHOD FOR ANALYZING A USER'S REACTION TO AT LEAST ONE STIMULUS

(71) Applicant: OSPEDALE SAN RAFFAELE S.R.L., Milano (MI) (IT)

(72) Inventors: Alberto SANNA, Milano (MI) (IT); Matteo ZARDIN, Milano (MI) (IT); Stela MUSTEATA, Milano (MI) (IT); Federica AGOSTA, Milano (MI) (IT); Massimo FILIPPI, Milano (MI) (IT)

(73) Assignee: OSPEDALE SAN RAFFAELE S.R.L., Milano (MI) (IT)

(21) Appl. No.: 19/131,035

(22) PCT Filed: Nov. 20, 2023
(86) PCT No.: PCT/IB2023/061677

§ 371 Date: May 19, 2025

(30) Foreign Application Priority Data
Nov. 21, 2022 (IT) 102022000023925

(51) Int. Cl. A61B 5/00

(52) U.S. Cl. CPC A61B 5/4088; A61B 5/7264; A61B 2562/02

(57) ABSTRACT

A method for analyzing a user's reaction to a stimulus is described which includes providing a user stimulus and a stimulation parameter through a sensory stimulator, measuring a biological response and acquiring a biosignal which indicates a neurophysiological reaction provides neurophysiological reaction data which generates sensory metadata indicative of the user's response to the corresponding stimulus. The sensory metadata is archived to obtain a plurality of sensory metadata associated with the user. Each piece of stored sensory metadata is processed according to input information, generating an output information including a reaction index to the stimulus as a function of the input information, and modifying a stimulation parameter according to the reaction index.

### TECHNICAL FIELD

The present invention relates to a method for analyzing one or more reactions to at least one stimulus provided to a user. The invention further relates to a computer program capable of performing the steps of the method as well as a computer-readable medium or an electronic device comprising a computer program for implementing the method. In addition, the invention relates to an apparatus for implementing the method of analyzing one or more reactions to at least one stimulus provided to a user, in particular for the diagnosis of a neurocognitive disorder.

### BACKGROUND ART

Currently, at least one fifth (19%) of the population of the European Union is older than 65 years. Population ageing is usually accompanied by an increased burden of disease, such as cardiovascular disease, diabetes, Alzheimer's, and other neurodegenerative diseases. Dementia is the fifth leading cause of death in the world. Specifically, epidemiological studies have shown that Alzheimer's, the most prevalent form of dementia, will affect 152 million individuals by 2050. In Italy, it is estimated that out of 1, 241,000 cases of dementia, about half (600,000) are Alzheimer's (XVIII Congresso Nazionale della Società Italiana di Riabilitazione Neurologica [EN: National Congress of the Italian Society of Neurological Rehabilitation], 2018).

Currently, the course of a diagnosis of a neurocognitive disease generally involves the use of techniques and technologies that are very expensive from an economic point of view (such as magnetic resonance imaging or PET) with long waiting times (depending on the national health system) and usually carried out in locations far from the patient's residence.

Neurocognitive diseases are characterized by several clinical manifestations that affect various aspects of an individual's nervous function, motor behaviour, and cognitive behaviour. These clinical manifestations can be identified on the basis of the individual's reaction to certain external stimuli. However, analyzing the reaction to these stimuli is usually carried out by qualified personnel and is not always objective, depending precisely on the interpretation of the personnel who carry out the analysis. In addition, the individual is required to go in person to specialized facilities to carry out the analysis.

Therefore, it is an object of the present invention to provide a method for analyzing the reaction of an individual to a series of external stimuli that can be carried out by anyone, even possibly by the same individual, and that has a high degree of objectivity.

### DISCLOSURE OF THE INVENTION

These objects are achieved by a method, by an apparatus, by a computer program and by a medium readable by a computer according to the claims at the end of the present description.

In one aspect of the invention a method is provided for analyzing a user's reaction to at least one stimulus, the method comprising:
providing at least one stimulus to the user selected from a plurality of stimuli according to an input information and at least one stimulation parameter from a plurality of stimulation parameters through one or more sensory stimulators;
measuring a biological response following the providing of said stimulus and acquiring at least one biosignal through one or more biometric sensors, wherein the biosignal indicates a neurophysiological reaction to the stimulus and comprises data of neurophysiological reaction;
associating the neurophysiological reaction data with the corresponding stimulus through at least one processor to generate at least one piece of sensory metadata indicative of the user's response to the corresponding stimulus;
archiving the sensory metadata within a memory support to obtain a plurality of sensory metadata associated with the user;
processing each piece of sensory metadata stored in the memory support through the processor according to the input information;
generating an output information following the processing of each piece of sensory metadata, the output information comprising a reaction index to the stimulus as a function of the input information; and
modifying at least one stimulation parameter according to the reaction index.

Through this method, one or more stimuli are presented to a human subject that elicit a sensory response by means of a stimulator. During stimulation, the biological response of the subject is measured by one or more biometric sensors which produce one or more biosignals. The biosignals recorded are reprocessed. Through this reprocessing, information about the subject's response to the stimulation is provided on the basis of the type of output desired by the stimulation itself, i.e. on the basis of the initial information. All of these operations are performed by one or more processors connected to the stimulator and biometric sensors.

Thus, information about an individual's reaction to one or more stimuli can be obtained through a reference index (reaction index) in an objective manner without necessarily requiring an expert to interpret the data or select the appropriate stimuli.

In a further aspect of the invention there is provided a computer program capable of performing the steps of the method of analysis described herein.

In a further aspect of the invention, there is provided a computer-readable medium or an electronic device comprising a computer program for implementing the method of analysis described herein.

In another aspect of the invention, there is provided an apparatus for implementing the method of analysis described herein, in particular for the diagnosis of a neurocognitive disorder, the apparatus comprising:
at least one sensory stimulator for providing at least one stimulus to the user selected from a plurality of stimuli according to input information and at least one stimulation parameter from a plurality of stimulation parameters;
at least one biometric sensor for measuring a biological response following the providing of said stimulus and acquiring at least one biosignal, wherein the biosignal indicates a neurophysiological reaction to the stimulus and comprises neurophysiological reaction data;
at least one processor connected to the sensory stimulator and to the biometric sensor for associating the neurophysiological reaction data with the corresponding stimulus to generate at least one piece of sensory metadata indicative of the response of the user to the corresponding stimulus; and
at least one memory support for archiving the sensory metadata and obtaining a plurality of sensory metadata associated with the user,
wherein the processor is configured to process each piece of sensory metadata stored in the memory support according to the input information, generate an output information following the processing of each piece of sensory metadata, the output information comprising a reaction index to the stimulus as a function of the input information, and to modify at least one stimulation parameter as a function of the reaction index.

Advantageously, the apparatus may be a dedicated system for an analysis of one or more reactions by the subject and employed medically in clinical settings by any type of personnel. On the other hand, the apparatus can also have applications other than medical, for example on the sports field or within companies, i.e. in all those applications in which an objective analysis of the reactions of an individual to one or more stimuli is required.

In addition, the apparatus may advantageously be a portable device, for example a cellular phone, which may be used directly by the individual themself to evaluate their reactions to certain stimuli.

These and other aspects of the present invention will become more apparent by reading the following description of some preferred embodiments disclosed below.

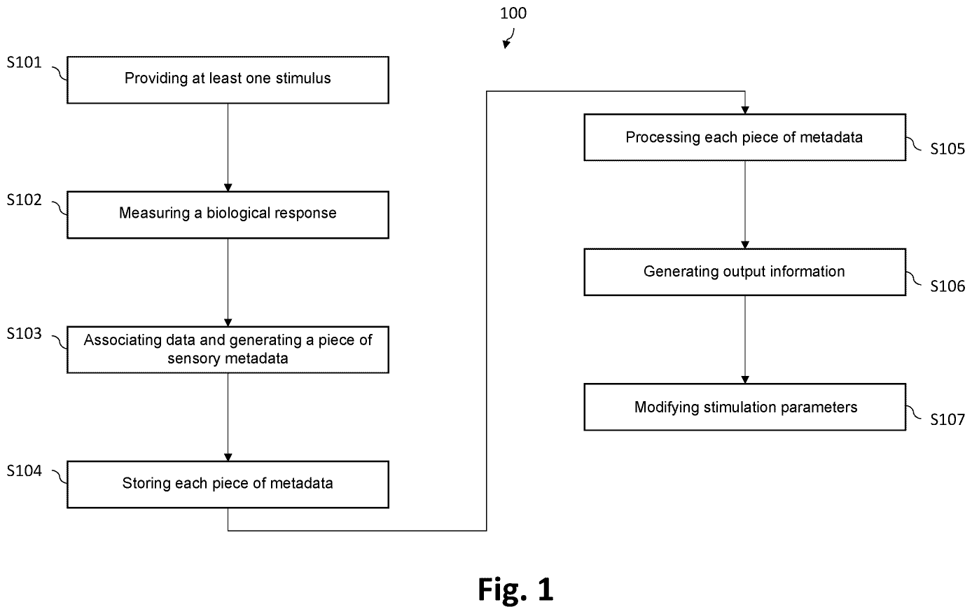

FIG. 1 shows a flow diagram of the method according to an example.

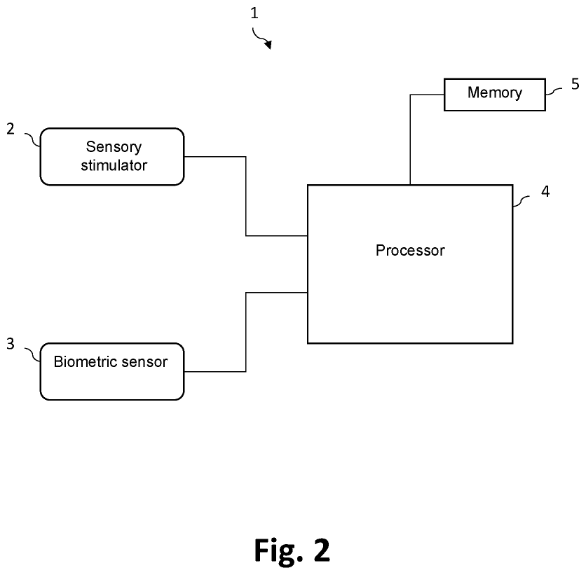

FIG. 2 shows a schematic representation of an apparatus according to an example.

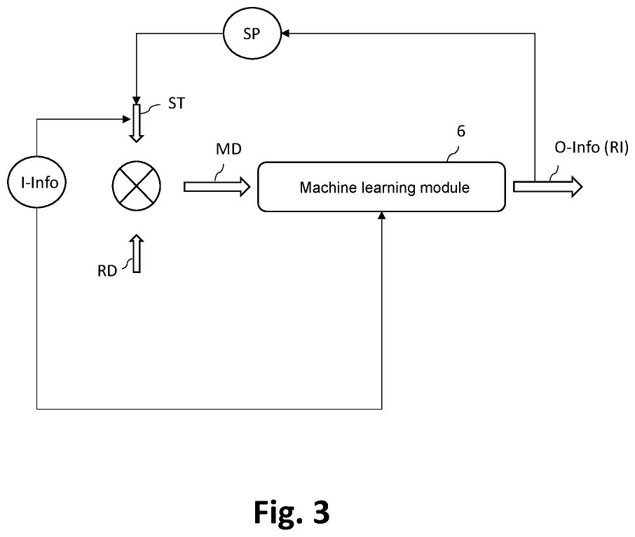

FIG. 3 shows a schematic representation of the processing of metadata according to an example.

FIG. 1 shows a flow chart describing the method 100 for analyzing one or more reactions by a user to one or more stimuli. By contrast, FIG. 2 shows a schematic representation of an apparatus 1 employed for implementing the method 100. Specifically, the apparatus 1 comprises at least one processor 4 to which one or more sensory stimulators 2, one or more biometric sensors 3 and one or more memory supports 5 are connected. FIG. 3 schematically shows the analysis of the signals and data processed according to the method 100.

Returning to FIG. 1, in step S101, at least one stimulus ST is provided to the user. The stimulus ST is selected from a plurality of stimuli of different nature or category.

In one example, the plurality of stimuli ST comprises stimuli ST generated by different sensory stimulators 2. For example, the plurality of stimuli may comprise visual stimuli (images or videos), auditory stimuli (sounds, music, or melodies), olfactory stimuli (aromas or scents), or a combination of these. Thus, according to one example, the sensory stimulator 2 may be a visual stimulator (e.g. projector screen), or an auditory stimulator (e.g. sound amplifier), or a taste stimulator (e.g. item to be tasted, food and drink), or an olfactory stimulator (e.g. odour diffuser), or a tactile-proprioceptive stimulator (e.g. item to be touched, fabrics and materials).

Alternatively or additionally, the plurality of stimuli ST may comprise stimuli ST associated or not associated with the personal lived experience of the user. For example, stimuli can be divided into two classes, those that pertain to the history of the subject and those that do not pertain to the personal history of the individual under analysis. The first, for example, are represented by digitizations of photographs, postcards, paintings etc. (or sounds of music) that have or have had a relevance in the personal emotional and cognitive history of the subject (e.g. a photo of their wedding, their favourite song). The latter, on the other hand, are digitizations of images or sounds that are not directly related to the subject's history but are extraneous as far as possible to their personal history (e.g. the cover of an album and the corresponding music not known to the subject themself).

As mentioned, the stimuli ST proposed to the subject during stimulation can be classified according to the traceability of the stimulus ST to the personal history of the subject. Highlighting and making this type of distinction allows the concept of stimulation personalization to be introduced, which is certainly independent of the functions that the apparatus 1 performs but which is functional for the use of the apparatus 1 for the purpose.

The plurality of stimuli may be stored within a stimulus database connected to the processor 4. The database can advantageously be organized to classify stimuli according to their nature (e.g. visual, olfactory, auditory, etc.) and their class (e.g. personal and non-personal stimuli).

The stimuli are selected according to input information I-Info and at least one stimulation parameter SP. The input information I-Info represents a kind of question that is to be answered as a result of the analysis of the reactions to the stimuli experienced by the user. In other words, once this input information I-Info is established, some stimuli are selected as they are considered more suitable for answering the question.

The selection of the stimuli also takes place as a function of at least one stimulation parameter SP. This parameter is selected from a plurality of stimulation parameters. In one example, the plurality of stimulation parameters SP comprises at least one of:
the total stimulation time;
the stimulation time with a single stimulus ST;
the number of stimuli ST;
the intensity of the stimulus ST;
the sequence with which the stimuli ST are provided; and
the typology of the stimulus ST.

This list of parameters is not exhaustive and other types of parameters may be considered.

In step S102, a biological response is measured following the providing of the stimulus ST and at least one biosignal is acquired by one or more biometric sensors 3. Specifically, the biosignal indicates a neurophysiological reaction to the stimulus ST and comprises neurophysiological reaction data RD. In one example, the biometric sensor 3 is configured to translate into numerical data a physical and/or chemical and/or mechanical reaction of a biological process of the user. For example, the biometric sensor 3 may be a PPG sensor (i.e. PhotoPlethysmoGraphic or sensor for photoplethysmography) which quantifies in numerical time series the information from the changes in blood volume in the body's vessels. The biometric sensor 3 may alternatively be a sensor for measuring blood pressure, heartbeat, heart rate, respiratory rate, electroencephalogram, electrocardiogram, oxygen saturation, skin sweat analysis, or temperature of the individual. Advantageously, the biometric sensor 3 may comprise an eye-gaze tracker.

The biometric sensor 3 may advantageously be applied to a part of the user's body and/or be wearable by the individual.

Different types of biometric sensors 3 are known for acquiring different types of biosignals. However, the apparatus 1 is to be understood as an apparatus capable of integrating and therefore interfacing with any set (i.e. subset or overset) of biometric sensors 3 thus acquiring a multitude of biosignals dependent on the biometric sensors 3 used.

Advantageously, the biometric sensors 3 employed in this method can be exported to the user's home environment so that the user themself can possibly apply the method to themself unaided, or can be helped by a person without particular medical skills.

In step S103, the processor 4 associates the corresponding stimulus ST with the neurophysiological reaction data RD to generate at least one piece of sensory metadata MD. This piece of metadata MD is indicative of the user's response to the corresponding stimulus ST. Each piece of sensory metadata MD generated is stored within a memory support 5 so as to obtain a plurality of sensory metadata MD associated with the user (step S104). In other words, the memory support 5 comprises the set of reactions experienced by the user to the various stimuli experienced.

In step S105, each piece of sensory metadata MD stored in the memory support 5 is processed by the processor 4 according to the input information, and in step S106, output information O-Info is generated following the processing of the sensory metadata MD. The output information O-Info comprises a stimulus reaction index RI as a function of the input information I-Info.

Based on the obtained reaction index RI, at least one of the stimulation parameters SP is changed (step S107). For example, the stimulus is changed within the same category (e.g., a different image), or the category of the stimulus is changed (e.g., from an image to a sound), or the class of the stimulus is changed (e.g., from a stimulus associated with a personal experience to a stimulus not known to the individual). Alternatively or additionally, the modification of the stimulation parameter SP may result in a change of the stimulus itself. It should be noted that a stimulus can be associated with a piece of stimulus data. For example, if the stimulus is an image (visual stimulus), the stimulus can be represented by an image file. If the stimulus is a sound, (sound stimulus), the stimulus can be represented by an audio file. Thus, a modification of the stimulus can be understood, in addition to a replacement with a different stimulus, also as a modification of the piece of stimulus data, for example a variation of the image file or the audio file. For example, an enlarged image, a more intense sound, a cropped image in a predefined region, etc. In other words, changing the stimulation parameters SP may mean a change of the stimulus data.

Thus, in addition to or as an alternative to manipulating at least one stimulation parameter, the method 100 described herein may comprise the step of manipulating and/or modifying the stimulus itself (e.g., the piece of stimulus data) as a function of the reaction index RI. The method 100 further comprises the possibility of providing again to the user a manipulated and/or modified stimulus in the same context or in a context different from the original one.

The manipulation and/or modification of the stimulus may be preceded by the identification of one or more salient elements of the stimulus for the individual user. The phrase “salient element” means a region or portion of the stimulus that has user-specific characteristics derived from the biometric sensors following the providing of the stimulus. For example, in the case of a visual stimulus such as an image, the salient element could be a portion of said image, e.g. the face of a person who elicited a particular reaction in the user as assessed by the biometric sensors. In the case of a sound stimulus, on the other hand, the salient element could be a portion of the audio file heard by the user. In other words, the salient element is a portion of a piece of stimulus data identified based on the user's reaction to the providing of the stimulus through the biometric sensors. It is evident that the salient element can be closely linked to the user and in particular to the personal experience of the user themself.

In one example, modifying at least one of the stimulation parameters SP is achieved by manipulating and providing again the stimulus to the user, wherein manipulating the stimulus comprises modifying one or more previously identified salient elements of the stimulus. In other words, manipulating the stimulus comprises modifying one or more previously identified portions of a piece of stimulus data. For example, the face of the person identified as a salient element within the visual stimulus shown to the user is provided again magnified within the same image or the audio portion is reproduced at a greater volume (the salient element is modified within the original stimulus).

Alternatively or additionally, manipulating the stimulus comprises removing one or more previously identified salient elements of the stimulus. In other words, manipulating the stimulus comprises removing one or more previously identified portions of a piece of stimulus data. For example, the face of the person identified as a salient element within the visual stimulus shown to the user is deleted from the same image or the audio portion is deleted or reproduced without volume (the salient element is eliminated from the original stimulus). Alternatively or additionally, manipulating the stimulus comprises moving one or more previously identified salient elements of the stimulus within a new stimulus. In other words, manipulating the stimulus comprises moving one or more previously identified portions of a piece of stimulus data within another stimulus. For example, the face of the person identified as a salient element within the visual stimulus shown to the user is provided again within a different image or the audio portion is reproduced within a different audio signal (the salient element is inserted within a stimulus different from the original).

The processor 4 is the component through which the apparatus 1 interfaces with the sensory stimulator 2 and the biometric sensors 3, reprocesses the recorded biosignals on the basis of the stimulation and produces information about the response of the subject to the stimulation. For example, by reprocessing the acquired signal by means of a PPG sensor via a static visual-type stimulation by means of a projector screen, the processor 4 can indicate the average heartbeat rate of the subject during the stimulation itself.

In one example, modifying at least one stimulation parameter SP occurs automatically by means of the processor 4. In this way, it is possible to modify stimuli or modify stimulus data without the intervention of a decision by another individual. This means that the analysis of the reaction to stimuli can be carried out by the individual quickly and objectively. For example, the apparatus 1 can be an electronic device of the user (mobile phone, tablet, laptop or computer) and the stimulus can be represented by one or more images or one or more sounds taken from the electronic device (for example associated with the user's personal experience) or directly from the web. The biometric sensor may be integrated into the apparatus (e.g., a camera for recognizing the user's facial expressions or a contact heart rate monitor) or connected thereto via cable or wirelessly. The processor 4 of the apparatus 1 is configured to process the biosignals and the neurophysiological reaction data RD and generate a piece of output information O-Info with a reaction index RI linked to the provided stimulus ST. The reaction index RI can be imagined as a piece of numerical data that can vary within a certain range of values. If the reaction index is within a certain range or is greater than or less than a certain threshold value, then the user's reaction may be considered to be in agreement or not in agreement with the input information (I-Info). This may involve a modification of the stimuli ST as mentioned above for further analysis or a final decision on whether or not the user's reaction is in agreement with the input information (I-Info). In the case of a modification of the stimuli ST, the analysis continues to evaluate a possible trend related to the user's reaction as to whether or not it is in agreement with the input information (I-Info). The stimulus parameters SP are for example modified to confirm this trend.

In one example, processing each piece of sensory metadata MD comprises using a machine learning (ML) process. In particular, the machine learning process may comprise one or more machine learning models trained prior to the providing of at least one stimulus ST to the user. Processing each piece of sensory metadata MD may further comprise extracting biosignal description indices used as input data in a machine learning module 6.

The biosignal reprocessing process operated by the processor 4 of the apparatus 1 consists essentially of two steps. In a first step, starting from the acquired biosignals, the processor 4 operates a real signal processing that, through a filtering step, allows indices to be obtained that synthesise and describe the behaviour. These indices are better known as signal “features”. Once these features are obtained, they are introduced into Machine Learning (ML) models chosen and previously and suitably trained in order to produce a result that responds to the desired output request.

The training of models ML takes place on the basis of data collected in a period of time prior to the apparatus 1 being employed for the use for which it is designed. In addition, this training can be updated over time by strengthening and making the set of models ML employed accurate.

The process of associating the stimuli ST (stimulus data) and the neurophysiological reaction data RD to obtain a piece of sensory metadata MD and processing the metadata MD in a machine learning module 6 to generate a reaction index RI is shown in FIG. 3.

It is noted that the processing which the processor 4 performs on the signals produces an output or output information O-Info. This output corresponds to the response, which the apparatus 1 itself provides, to the question with which the apparatus 1 is queried. This response depends substantially on the type of stimulation used (not only on the type of nature of the stimulation but also on the procedural paradigm with which the stimuli are presented) and on the type of reprocessing of the biosignals that the apparatus 1 operates. It is recalled that this reprocessing of the biosignals depends to a large extent on the models ML adopted.

The apparatus 1 may be used for different purposes, i.e. to obtain different types of output. In one example, the apparatus 1 can be applied in the clinical setting. For example, the apparatus 1 may be used for a diagnosis of neurocognitive disorders. The apparatus 1 can also be used with a theragnostic approach, that is, also to treat a user with a neurocognitive disorder, by providing stimuli with a therapeutic effect according to the recorded biosignals. In particular, the apparatus may advantageously be used for the early detection of Alzheimer's disease. Therefore, the above-described method can be used as a diagnosis method for early detection of Alzheimer's disease. In this case, the question for which an output from apparatus 1 is desired (the input information I-Info) corresponds to the request for which, through a precise process and a precise procedure of static visual stimulation (e.g. images), it is possible to identify early and with what probability the conversion of a subject from the state of amnestic Mild Cognitive Impairment (aMCI) to that of Alzheimer's Disease (AD).

Initially, one or more stimuli ST eliciting a sensory response by a sensory stimulator 2 is presented to a human subject. As described above, the human subject to whom the apparatus 1 presents the stimuli may be a subject diagnosed with aMCI. The sensory stimulator 2 is represented by a screen on which images appear with a sequence and timing that is managed by the processor 4 of the apparatus 1 on instruction given by an operator or on the basis of predefined parameters within the apparatus 1. The operator can set and adjust the stimulation parameters SP of the apparatus 1 (e.g. total stimulation time, stimulation time with the single stimulus, number of stimuli, order and logic with which the stimuli are presented, etc.). However, the apparatus 1 can autonomously adjust the stimulation parameters SP by customizing them for each subject and increasing the accuracy of the results without the intervention of an operator. As described above, the stimuli ST that are presented to subjects are images that can be divided into two distinct classes, stimuli pertaining to the subject's history and stimuli that do not pertain to the subject's personal history.

Stimulation occurs as a continuous presentation of images, that is, one stimulus ST after another with a stimulus ST display time that varies from stimulus to stimulus. During the stimulation step, several biometric sensors 3 integrated with acquire biosignals from the subject. In the apparatus 1 particular, the biometric sensors 3 used make it possible to acquire the photoplethysmographic (PPG) biosignals, galvanic skin response (GSR) biosignals, electroencephalographic (EEG) biosignals, eye movement (eye tracking, ET) biosignals, and facial expression (facial expression recognition, FER) biosignals. Again, the number of integrated biometric sensors 3 is not limited to those for the detection of the above biosignals, but may be extended to biometric sensors 3 for the detection of further biosignals.

During stimulation, the apparatus 1 measures the biological response of the subject by means of one or more biometric sensors 3 which produce one or more biosignals. Any type of sensory stimulation on a subject produces a biological response (e.g. variation in the biological state of the subject) that is measurable by the type of biometric sensor 3 that is able to evaluate the variations and is a function of the elicitation that the stimulation itself produces on the subject. The imminent result of this multiple measurement consists in the production of a multitude of numerical time series (e.g. biosignals), as many as the biometric sensors 3 employed. The apparatus 1 stores and associates the temporarily recorded biosignals to the corresponding stimulus ST presented during the stimulation and personally to the stimulated subject. Then, each stored biosignal is ready to be reprocessed by the processor 4.

It is noted that a reprocessing of a biosignal is to be understood s a reprocessing of sensory metadata (MD) comprising said biosignal.

The reprocessing of the recorded biosignals serves two purposes. First of all, it allows the value of some indices (e.g. features) to be calculated, which coincide with synthetic descriptors of the characteristics of the biosignals (e.g. average of the signal, maximum value of the signal, Lyapunov exponent of the signal, average frequency of the signal, etc.). The apparatus 1, therefore, computationally extracts a multitude of features from each signal. The extraction process carried out by the apparatus 1 can be described with the concatenation of two operations. The first involves the filtering of the biosignal (e.g. reduction of signal noise) and the second involves the application of statistical and typical methods of digital filtering of the signals that allow, finally, the desired features to be obtained.

Once the features are extracted, the apparatus 1 passes them in input to a previously trained model ML that provides an output that coincides with the probability that the model ML estimates that the subject will develop AD. It is necessary to note that the training of the model ML takes place before it is imported into the apparatus 1. Training is performed based on previously acquired data with the same stimulation paradigm.

Through such reprocessing, the apparatus 1 provides information about the response of the subject to the stimulation based on the type of output desired from the stimulation itself. As described, the information (O-Info) that the device returns coincides with the response to the question (I-Info) for which it is employed. In this case, it is desired to know if the biological condition of the aMCI subject suggests a condition of possible conversion to AD. The output that the device provides is not to be considered as a diagnosis, but rather as information to support the clinical decision. The output comprises a numerical value (the reaction index RI) that indicates the likelihood that the subject will become an AD subject.

The apparatus 1 uses a processor 4 to perform all the operations described above. All the operations carried out by the apparatus 1 are managed by a dedicated processor 4 to which the biometric sensors 3 and the stimulation screen 2 are connected. Signal reprocessing and output generation takes place using the same dedicated processor 4.

As mentioned above, with the method 100 described herein it is possible to select and/or modify stimuli ST (stimulus data) in an automated manner.

First, the so-called salient elements (ES) of each stimulus ST for the individual user are automatically identified. “Salient elements” refer to those elements that, during the providing of the stimulus itself, have a combination of characteristics obtained from biometric sensors. Specifically, if a visual stimulus is considered, the signals and the resulting extracted characteristics that are expected to contribute most to the identification of the salient elements of a stimulus can be, for example, the eye-tracker signal (focus of the user's eye movements), the galvanic response of the skin (high electrodermal activity) and/or the identification of an expressive configuration of the face connoting a positive or negative (i.e. non-neutral) emotion.

To understand how the modification of the stimulus and the identification of the salient elements occurs, the concept of a multi-parametric significance model of the recorded signals can be introduced.

For example, suppose that the user using the described method is equipped with a sensor dashboard for acquiring biomedical signals. From these signals, the processing system automatically extracts characteristics that may coincide with the signal itself or be its processing (e.g. characteristics extracted through the appropriate signal processing methods) or a combination with other signals.

For each signal and/or characteristic, certain thresholds are predetermined, which discretize the signal data and/or characteristic data itself, identifying states of significance of each. For example, the range of values that each signal can assume can be divided into three levels: high significance, medium significance and low significance. Thus, by assuming a sensor dashboard that allows for the acquisition and processing of six signals, it is possible to construct a 6×3 sized matrix. This matrix, updated at each timestamp, represents the configuration of the values of the signals discretized through the values 1 and 0.

TABLE 1
Significance matrix

| | Signal 1 | Signal 2 | Signal 3 | Signal 4 | Signal 5 | Signal 6 |
|---|---|---|---|---|---|---|
| High | 0 | 1 | 1 | 0 | 1 | 0 |
| Significance | | | | | | |
| Medium | 0 | 0 | 0 | 1 | 0 | 0 |
| Significance | | | | | | |
| Low | 1 | 0 | 0 | 0 | 0 | 1 |
| Significance | | | | | | |

As shown in Table 1, the entries of the significance matrix are 0 or 1 depending on the specific value assumed by the individual signal and/or characteristic in relation to its pre-established significance thresholds. Note that a high significance value indicates a particularly favourable condition for identifying a salient element for the subject. Each signal is assigned a weight and each level of significance for each signal is assigned a value. These values represent the importance of the signal in providing information on the physiological-emotional-cognitive state of the user.

By assigning a weight to each level of significance for each signal, the above matrix transforms into a significance matrix as shown in Table 2.

TABLE 2
Modified significance matrix

| | Signal | Signal | Signal | Signal | Signal | Signal |
|---|---|---|---|---|---|---|
| | 1 | 2 | 3 | 4 | 5 | 6 |
| High | 0 × 1.2 | 1 × 1.5 | 1 × 1.4 | 0 × 1.2 | 1 × 2.1 | 0 × 0.8 |
| Significance | | | | | | |
| Medium | 0 × 0.8 | 0 × 1.1 | 0 × 0.9 | 1 × 0.7 | 0 × 1.7 | 0 × 0.5 |
| Significance | | | | | | |
| Low | 1 × 0.2 | 0 × 0.8 | 0 × 0.5 | 0 × 0.4 | 0 × 1.4 | 1 × 0.2 |
| Significance | | | | | | |

Once the values of the individual items of the significance matrix are obtained, they are summed by providing a numerical value, called overall significance (SC). It should be noted that the overall significance varies according to the subject considered and its value is compared with a reference value, i.e. the activation threshold level. If SC exceeds the preset threshold, a salient element is identified.

Once the salient elements are extracted from the original stimulus, they will be provided again into a new stimulus, which will then be manipulated and/or modified with respect to the original, through the use of an artificial intelligence (AI) algorithm. The AI algorithm can automatically make several changes, such as providing again a stimulus equal to the original with changes applied to only the salient elements (e.g., change of position, intensity, size, etc.), applying changes to the entire image excluding the salient elements, or bringing these elements back into a new stimulus that proposes a different context from the original. In the specific case, the last modification is the one most favourable to the development of the familiarity characteristic.

These stimuli can be used both in the diagnostic step for the detection of the presence of disease, and as target elements for non-pharmacological neurocognitive intervention treatments. In the treatment, the different complexity of manipulation of the stimulus by the AI algorithm can be exploited, starting from a low manipulation and working up to a substantial manipulation. This manipulation can be continuously varied based on the performance of the individual user and, in general, the progress of the treatment.

The possibility of selecting and modifying stimuli, with consequent personalization, is very useful in the investigation of different neurodegenerative conditions, both in the diagnosis and therapy steps. In fact, each condition of cognitive impairment is described by the presence of one or more features that characterize it and that, if properly investigated, can provide relevant information about the presence or absence of the same.

For example, if we consider Alzheimer's disease, it is possible to identify ‘familiarity’ as such a characteristic. The concept of familiarity reflects a sense of knowledge of the object without being able to specify its contextual, spatial and temporal details. The concept of familiarity applied to a stimulus, a person or an event has a generic, decontextualized meaning that a stimulus, or part of it, has been seen before.

From the earliest stages, Alzheimer's disease affects brain regions, such as the perirhinal and entorhinal cortex, which are critical for both spontaneous recovery, a process that relies on specific contextual and spatio-temporal information, and familiarity. Based on neuropathological studies, it is known that in these regions the first pathological neurofibrillary tangles are formed, which are the primary markers of the disease and which determine the onset of neurodegeneration. As a result, these same brain areas are already affected in patients with mild cognitive impairment (MCI) destined to develop Alzheimer's dementia, who therefore have difficulties in both spontaneous recovery and familiarity.

Until now, using standard approaches, it has not always been possible to study the feeling of familiarity in these patients, often due to the simplicity of the methodological approaches, which are unable to detect the presence of subtle differences between groups. The use of the proposed method, including the possibility of using stimuli selected and manipulated on the basis of subjective experience, is expected to determine familiarity in healthy subjects, as opposed to subjects destined to develop dementia, thus allowing the two groups to be differentiated.

The changes that can be applied to the original stimulus are multiple and vary on a scale that has different levels of complexity, where the minimum level does not introduce any change to the stimulus, while the maximum level makes a substantial change to the original stimulus (such as the creation of a new stimulus). The level of modification to be made is chosen, on a case-by-case basis, considering the physiological characteristics of the individual subject and the neurodegenerative condition to be evaluated.

Again, in the example of Alzheimer's disease, it is assumed that the feeling of familiarity can be effectively investigated by proposing a stimulus in which the salient elements, automatically identified on the basis of the specific physiological dynamics of the subject, have been manipulated and/or modified and re-proposed in contexts other than the original one.

It is noted that the disclosed method is particularly suitable for the study of pathological mechanisms underlying a given syndrome. Specifically, the approach of the present method is to investigate the specific brain circuits primarily affected in Alzheimer's disease from the early stages and extends to extra brain circuits that could be involved in the more advanced stages of the disease. However, by changing the paradigm proposed to the user, this approach can easily be applied for the analysis of other brain circuits that underlie pathophysiological mechanisms different from Alzheimer's disease. This method allows the recording of the user's physiological signals even when the user is not performing any task (i.e. in passive mode). This makes it possible to acquire information even in patients with neurodegenerative diseases (e.g. Alzheimer's) and language production problems (in addition to amnesic disorders), who would therefore be unable to speak.

A person skilled in the art may perform numerous further modifications and variations to the method and apparatus described above, in order to satisfy further and contingent requirements, all said modifications and variations being however included within the scope of protection of the present invention as defined by the appended claims.

1. A method for analyzing a user's reaction to a stimulus, the method comprising:
providing a stimulus to the user selected from a plurality of stimuli according to an input information and a stimulation parameter from a plurality of stimulation parameters through one or more sensory stimulators;
measuring a biological response following the providing of said stimulus and acquiring a biosignal through one or more biometric sensors, wherein the biosignal indicates a neurophysiological reaction to the stimulus and comprises data of neurophysiological reaction data;
associating the neurophysiological reaction data with the corresponding stimulus through a processor to generate a piece of sensory metadata indicative of the user's response to the corresponding stimulus;
archiving the sensory metadata within a memory support to obtain a plurality of sensory metadata associated with the user;
processing each piece of sensory metadata stored in the memory support through the processor according to the input information;
generating an output information following the processing of each piece of sensory metadata, the output information comprising a reaction index to the stimulus as a function of the input information, and
and modifying a stimulation parameter according to the reaction index.

2. The method according to claim 1, wherein the plurality of stimulation parameters comprises at least one of:
the total stimulation time;
the stimulation time with a single stimulus;
the number of stimuli;
the intensity of the stimulus;
the sequence with which the stimuli are provided; and
the typology of the stimulus.

3. The method according to claim 1, wherein modifying the stimulation parameter occurs automatically by means of the processor.

4. The method according to claim 1, wherein the plurality of stimuli comprises:
a. stimuli generated by different sensory stimulators; and/or
b. stimuli associated or not associated with the personal lived experience of the user.

5. The method according to claim 1, further comprising modifying the stimulus as a function of the reaction index and providing again to the user said modified stimulus in the same context or in a different context.

6. The method according to claim 5, wherein the modification of the stimulus is preceded by the identification of a salient element of the stimulus for the user, wherein the salient element of the stimulus is a region or portion of the stimulus that has user-specific characteristics derived from the biometric sensors following the providing of the stimulus.

7. The method according to claim 6, wherein each stimulus is associated with a piece of stimulus data and the salient element represents a portion of the piece of stimulus data identified on the basis of the user's reaction to the providing of the stimulus through the biometric sensors.

8. The method according to claim 6, wherein modifying of the stimulation parameters comprises manipulating and providing again the stimulus to the user, wherein:
a. manipulating the stimulus comprises modifying one or more previously identified salient elements of the stimulus and/or
b. manipulating the stimulus comprises removing one or more previously identified salient elements of the stimulus; and/or
c. manipulating the stimulus comprises moving one or more previously identified salient elements of the stimulus within a new stimulus.

9. The method according to claim 1, wherein the processing each piece of sensory metadata comprises using a machine learning process.

10. The method according to claim 9, wherein the machine learning process comprises one or more machine learning models trained prior to the providing of a stimulus to the user.

11. The method according to claim 1, wherein processing each piece of sensory metadata comprises extracting biosignal description indices used as input data into a machine learning module.

12. A computer program capable of performing the steps of the method of claim 1.

13. A computer readable medium or electronic device comprising a computer program for implementing the method according to claim 1.

14. An apparatus for implementing the method according to claim 1, for the diagnosis of a neurocognitive disorder, the apparatus comprising:
a sensory stimulator for providing a stimulus to the user selected from a plurality of stimuli as a function of input information and a stimulation parameter from a plurality of stimulation parameters;
a biometric sensor for measuring a biological response following the providing of said stimulus and acquiring a biosignal, wherein the biosignal indicates a neurophysiological reaction to the stimulus and comprises neurophysiological reaction data;
a processor connected to the sensory stimulator and to the biometric sensor for associating the neurophysiological reaction data with the corresponding stimulus to generate a piece of sensory metadata indicative of the response of the user to the corresponding stimulus; and a memory support for storing the sensory metadata and obtaining a plurality of sensory metadata associated with the user,
wherein the processor is configured to process each piece of sensory metadata stored in the memory support according to the input information, generate an output information following the processing of each piece of sensory metadata, the output information comprising a reaction index to the stimulus as a function of the input information, and to modify a stimulation parameter as a function of the reaction index.

15. The apparatus according to claim 14, wherein:
a. the sensory stimulator is a visual stimulator, or an auditory stimulator, or a taste stimulator, or an olfactory stimulator, or a tactile-proprioceptive stimulator; and/or
b. the biometric sensor is configured to translate into numerical data a physical and/or chemical and/or mechanical reaction of a biological process of the user.
